(19) United States
(12) Patent Application Publication
(10) Pub. No.: US 2020/0121163 A1
(43) Pub. Date: Apr. 23, 2020

(54) FLEXIBLE TUBE INSERTION APPARATUS, INSERTION CONTROL APPARATUS, AND FLEXIBLE TUBE INSERTION SUPPORT METHOD

(71) Applicant: OLYMPUS CORPORATION, Tokyo (JP)

(72) Inventor: Takeshi TAKAHASHI, Hachioji-shi (JP)

(73) Assignee: OLYMPUS CORPORATION, Tokyo (JP)

(21) Appl. No.: 16/722,052

(22) Filed: Dec. 20, 2019

Related U.S. Application Data
(63) Continuation of application No. PCT/JP2017/023390, filed on Jun. 26, 2017.

(51) Int. Cl. A61B 1/005; A61B 1/00; A61B 1/31; A61B 5/06

(52) U.S. Cl. CPC A61B 1/0051; A61B 1/00006; A61B 2090/064; A61B 1/31; A61B 5/062; A61B 1/0058

(57) ABSTRACT

An insertion apparatus includes an insertion section including a flexible tube section configured to passively bend, and a bendable section connected to the flexible tube section on a distal end side and configured to actively change its bend shape, and a characteristic changer provided in the flexible tube section and configured to change a force quantity acting on a distal end of the insertion section from an insertion target body. The apparatus also includes an analyzer configured to calculate information relating to a deformation force quantity that restores bending of the insertion section on the proximal end side of the changer, and a controller configured to control the changer based on the information relating to the deformation force quantity.

### CROSS-REFERENCE TO RELATED APPLICATIONS

This application is a Continuation Application of PCT Application No. PCT/JP2017/023390, filed Jun. 26, 2017, the entire contents of which are incorporated herein by reference.

### BACKGROUND OF THE INVENTION

### 1. Field of the Invention

The present invention relates to a flexible tube insertion apparatus including a flexible tube section to be inserted into an insertion target body, an insertion control apparatus, and a flexible tube insertion support method.

### 2. Description of the Related Art

In a flexible tube insertion apparatus such as an endoscope apparatus, it is known that a bending stiffness of an insertion section is partly changed in order to enhance the insertability of the insertion section (flexible tube section).

For example, Japanese Patent No. 5851139 discloses an endoscope apparatus that includes an insertion section including a first bendable section and a second bendable section that are actively bendable. The second bendable section is provided with an adjuster capable of changing its bending stiffness. In this apparatus, the bending stiffness of the second bendable section is changed by a surgeon's judgment or by a proper setting, so that the second bendable section may become easily bendable or less easily bendable, and therefore the insertability is enhanced.

For example, Jpn. Pat. Appln. KOKAI Publication No. 2016-7434 discloses an endoscope apparatus in which an insertion section is divided into segments in the longitudinal direction, and the shape of each segment is detected. By varying the bending stiffness of each section in accordance with the detected bend shape, the insertability is enhanced.

### BRIEF SUMMARY OF THE INVENTION

According to an embodiment of the present invention, a flexible tube insertion apparatus includes: an insertion section having a distal end and a proximal end, and including a flexible tube section configured to passively bend, and a bendable section that is connected to the flexible tube section on a distal end side of the flexible tube section and configured to actively change its bend shape, the insertion section being to be inserted into an insertion target body from the distal end side; a flexible tube characteristic changer that is provided in the flexible tube section and configured to change a force quantity acting on the distal end of the insertion section from the insertion target body, by changing a characteristic of the flexible tube section; a force quantity analyzer configured to calculate information relating to a deformation force quantity that is force that restores bending of the insertion section on the proximal end side with respect to the flexible tube characteristic changer; and a flexible tube characteristic controller configured to control the flexible tube characteristic changer, based on the information relating to the deformation force quantity.

According to another embodiment of the present invention, an insertion control apparatus includes: a force quantity analyzer configured to calculate information relating to a deformation force quantity that is force that restores bending of an insertion section on a proximal end side with respect to a flexible tube characteristic changer, the insertion section having a distal end and a proximal end, including a flexible tube section configured to passively bend, and a bendable section that is connected to the flexible tube section on a distal end side of the flexible tube section and configured to actively change its bend shape, and being to be inserted into an insertion target body from the distal end side, the flexible tube characteristic changer being provided in the flexible tube section and configured to change a force quantity acting on the distal end of the insertion section from the insertion target body, by changing a characteristic of the flexible tube section; and a flexible tube characteristic controller configured to control the flexible tube characteristic changer, based on the information relating to the deformation force quantity.

According to still another embodiment of the present invention, a flexible tube insertion support method includes: inserting an insertion section into an insertion target body, the insertion section having a distal end and a proximal end, and including a flexible tube section configured to passively bend, and a bendable section that is connected to the flexible tube section on a distal end side of the flexible tube section and configured to actively change its bend shape; changing a force quantity acting on the distal end of the insertion section from the insertion target body, by changing a characteristic of the flexible tube section; and calculating information relating to a deformation force quantity that is force that restores bending of the insertion section on the proximal end side with respect to a location where the characteristic of the flexible tube section is changed. The changing the force quantity acting on the distal end of the insertion section is executed based on the information relating to the deformation force quantity.

Advantages of the invention will be set forth in the description that follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view schematically showing an example of a flexible tube insertion apparatus according to a first embodiment.

FIG. 2 is a view showing an example of a distal end of an insertion section of the flexible tube insertion apparatus.

FIG. 3 is a view schematically showing an example of the insertion section of the flexible tube insertion apparatus including a bend shape detection device.

FIG. 4 is a block diagram showing an example of the flexible tube insertion apparatus according to the first embodiment.

FIG. 5 is a view schematically showing an example of a flexible tube characteristic changer.

FIG. 6 is a view showing an example of a voltage-bending stiffness characteristic of the flexible tube characteristic changer.

FIG. 7 is an anatomical drawing schematically showing parts of the large intestine.

FIG. 8 is a schematic view showing an example of insertion of a colonoscope by a colon shortening insertion method.

FIG. 9 is a view showing an example of the insertion state of the insertion section by the colon shortening insertion method.

FIG. 10 is a view showing an example of the insertion state of the insertion section by the colon shortening insertion method.

FIG. 11 is a view for explaining a concrete therapy technique in the insertion of a colonoscope by the colon shortening insertion method.

FIG. 12 is a view showing an example of the insertion state of the insertion section by the colon shortening insertion method.

FIG. 13 is a view showing an example of colonoscope insertion according to the first embodiment.

FIG. 14 is a view showing an example of an insertion support control flow according to the first embodiment.

FIG. 15 is a view for explaining bend shape information of an insertion section, which is acquired by a shape acquisition device.

FIG. 16 is a view showing an example of the relationship between a detection force quantity and an analysis force quantity.

FIG. 17 is a view showing an example of a bending stiffness change of the flexible tube characteristic changer.

FIG. 18 is a view showing an example of the bending stiffness change of the flexible tube characteristic changer.

FIG. 19 is a view showing another example of the insertion support control flow in the first embodiment.

FIG. 20 is a block diagram showing an example of a flexible tube insertion apparatus according to a second embodiment.

FIG. 21 is a view showing an example of an insertion support control flow according to the second embodiment.

FIG. 22 is a block diagram showing an example of a flexible tube insertion apparatus according to a third embodiment.

FIG. 23 is a view showing an example of an insertion support control flow according to the third embodiment.

FIG. 24 is a view schematically showing an example of an insertion section according to a fourth embodiment.

FIG. 25 is a view showing an example of a bend state of the insertion section according to the fourth embodiment.

### DETAILED DESCRIPTION OF THE INVENTION

Hereinafter, embodiments of the present invention will be described with reference to the accompanying drawings. In the description below, an endoscope apparatus is described as an example of a flexible tube insertion apparatus of the invention.

### First Embodiment

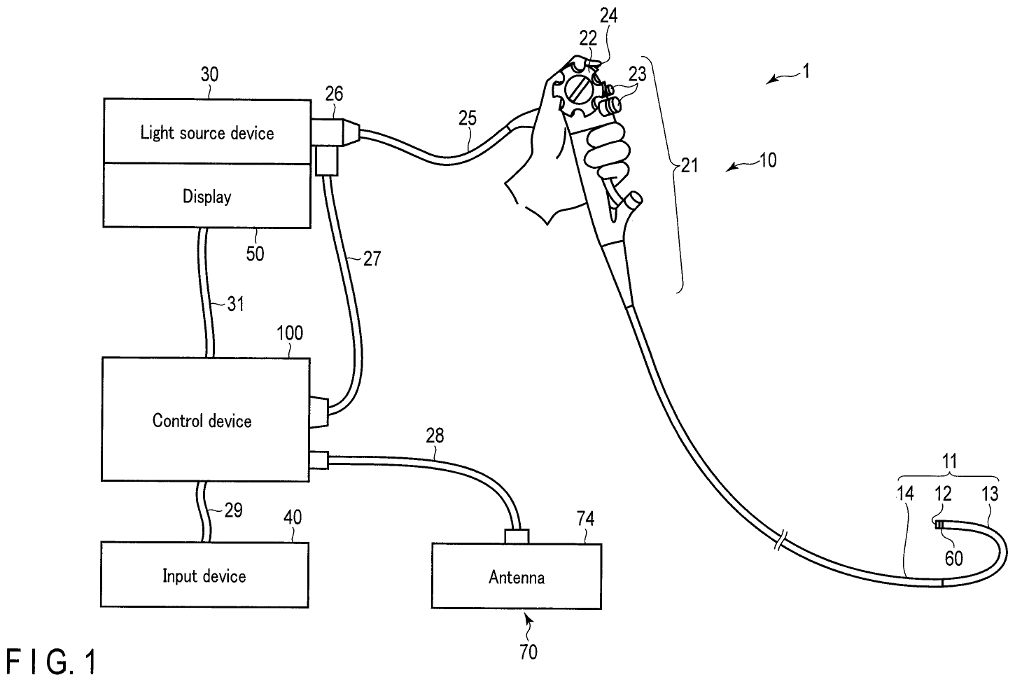

FIG. 1 is a view schematically showing an example of an endoscope apparatus 1. The endoscope apparatus 1 includes an endoscope 10, a light source device 30, an input device 40, a display 50, and a control device 100.

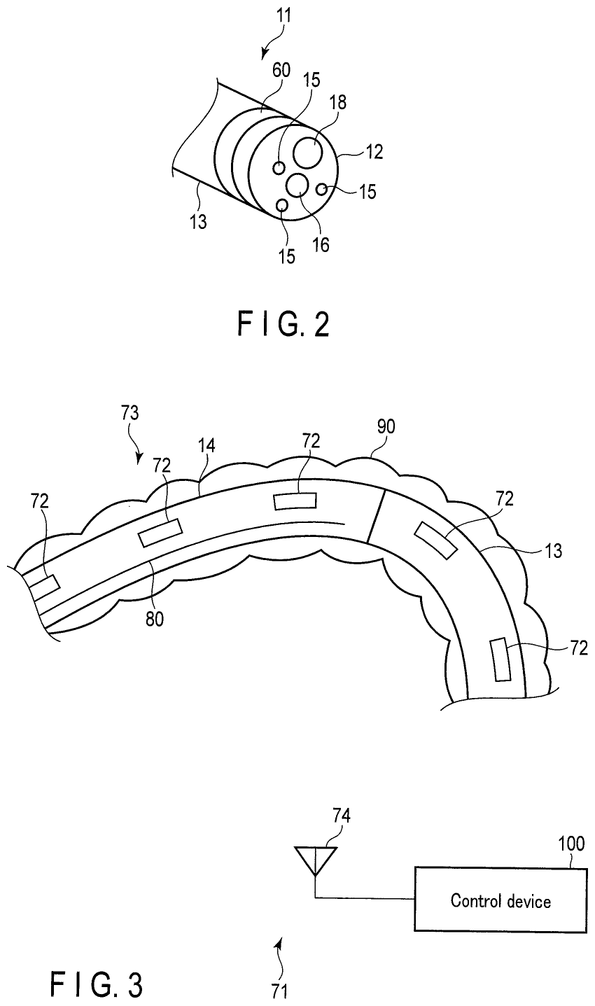

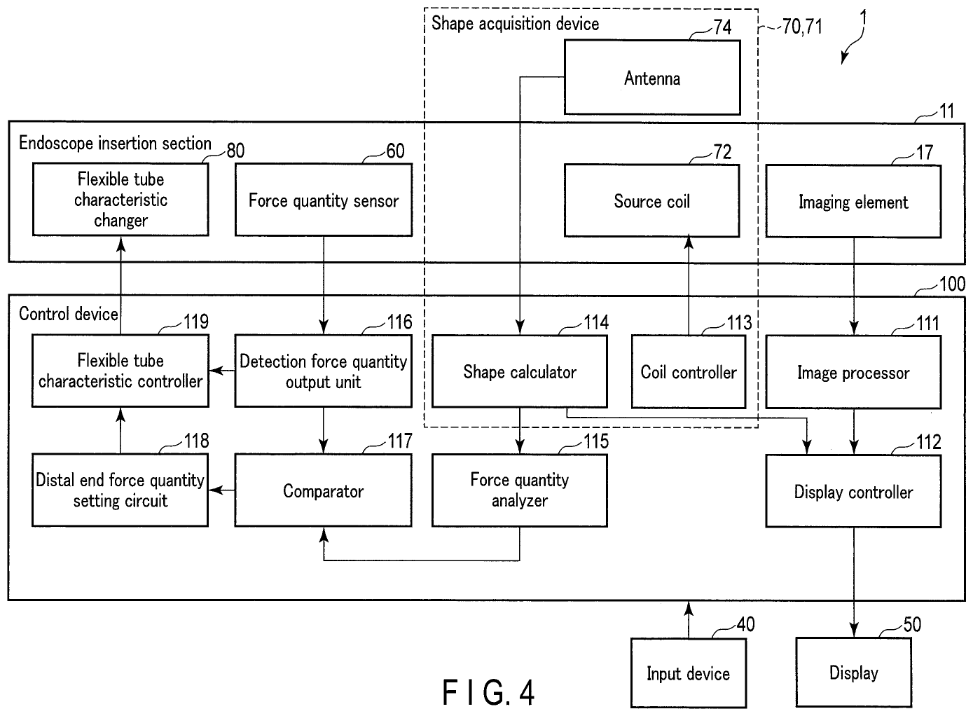

The endoscope 10 includes a tubular insertion section 11 to be inserted into an insertion target body, and a control section 21 provided on a proximal end side of the insertion section 11. The insertion section 11 includes a distal end hard section 12, a bendable section 13 provided on a proximal end side of the distal end hard section 12, and a flexible tube section 14 provided on a proximal end side of the bendable section 13. The distal end hard section 12 includes an illumination optical system shown in FIG. 2, which includes an observation optical system shown in FIG. 2 including illumination lenses 15 and an objective lens 16, and an imaging element 17 shown in FIG. 4, and the like. The bendable section 13 is a portion that is bent by an operation of the control section 21, and is configured to actively change its bend shape. The flexible tube section 14 is an elongated tubular portion with flexibility, and bends passively. The control section 21 is provided with an angle knob 22 that is used for a bending operation of the bendable section 13, and one or more buttons 23 that are used for various operations including air feeding, water feeding, and suction operations. By a surgeon operating the angle knob 22, the bendable section 13 is bent in a freely selected direction. In addition, the control section 21 is provided with one or more switches 24 to which functions, such as still/record of endoscopic images and focus switching, are assigned by the setting of the control device 100.

As shown in FIG. 1 and FIG. 2, a force quantity sensor 60 functioning as an external force detector is provided at a distal end of the bendable section 13. The force quantity sensor 60 is disposed, for example, on an outer peripheral surface of the bendable section 13. The force quantity sensor 60 detects external force applied to the distal end of the bendable section 13, i.e. the quantity of force acting on the distal end of the bendable section 13. The external force applied to the bendable section 13 is, for example, a contact force received from an insertion target body when the bendable section 13 comes in contact with the insertion target body.

The endoscope apparatus 1 includes a shape acquisition device 70. FIG. 3 is a view schematically showing an example of the insertion section 11 of the endoscope apparatus 1 including a bend shape detection device 71 of a magnetic sensor type, as an example of the shape acquisition device 70. In FIG. 3, the insertion section 11 is illustrated in a state in which the insertion section 11 is inserted into a bending insertion target body 90. The bend shape detection device 71 includes a source coil array 73 comprising source coils 72 for use in detecting a bend shape (bend angle, bend quantity, curvature or radius of curvature, or the like) of the insertion section 11.

In the description below, since the distal end hard section 12 is a very short portion in the entire length of the insertion section 11, unless explicitly indicated otherwise, it is assumed that the “insertion section 11” designates the bendable section 13 and flexible tube section 14. Specifically, unless explicitly indicated otherwise, the “insertion section” is used in substantially the same meaning as a bendable flexible tube in the flexible tube insertion apparatus. The “bend shape of the insertion section 11”, which is detected by the bend shape detection device 71, designates the bend shape of the bendable section 13 and flexible tube section 14, and the “distal end of the insertion section 11” is used in substantially the same meaning as the distal end of the bendable section 13.

The source coil 72 is a magnetic field generating element configured to generate a magnetic field. In the source coil array 73, the respective source coils 72 are arranged in the bendable section 13 and flexible tube section 14 at intervals in a longitudinal direction (axial direction) of the insertion section 11, in order to detect a bend shape in the longitudinal direction (axial direction) of the insertion section 11. FIG. 3 shows a configuration in which the source coils 72 are installed in the insertion section 11 in advance. However, a probe in which source coils are built may be inserted through a channel (communicating with a forceps hole 18 shown in FIG. 2) extending in the longitudinal direction in the insertion section 11.

The bend shape detection device 71 includes an antenna 74 for detecting a magnetic field generated by the source coils 72. The antenna 74 is a component separate from the endoscope 10, and is disposed around the insertion target body 90 into which the endoscope 10 is inserted. The antenna 74 is connected to the control device 100.

Referring back to FIG. 1, the light source 30 is connected to the endoscope apparatus 10 through a cable connector 26 at a distal end of a universal cable 25 extending from the control section 21. The universal cable 25 includes a light guide connected to the above-described illumination optical system, and a transmission cable connected to the imaging element 17. The light source device 30 includes a general light-emitting element such as a laser diode (LD), a light-emitting diode (LED), or the like. The light source device 30 supplies, through the light guide, illumination light that is radiated from an illumination window of the distal end hard section 12. Dimming or the like of the illumination light of the light source device 30 is controlled by the control device 100.

FIG. 4 is a block diagram showing an example of the endoscope apparatus 1 in the first embodiment. In FIG. 4, the light source device 30 is omitted. The control device 100 includes an image processor 111, a display controller 112, a coil controller 113, a shape calculator 114, a force quantity analyzer 115, a detection force quantity output unit 116, a comparator 117, a distal end force quantity setting circuitry 118, and a flexible tube characteristic controller 119. As shown in FIG. 1, the control device 100 is connected to the endoscope 10 and light source device 30 through the cable connector 26 and a cable 27. The control device 100 is also connected to the antenna 74 through a cable 28.

The above-described components of the control device 100 may be constituted by processors such as CPUs. In this case, for example, various programs for causing the processors to function as the respective components are prepared in an internal memory or external memory, which is not shown, and the processors implement the functions of the components of the control device 100 by executing the programs. Alternatively, the components of the control device 100 may be constituted by hardware circuitry including an ASIC (Application Specific Integrated Circuit), FPGA (Field-Programmable Gate Array), and the like.

The components of the control device 100, in particular, the shape calculator 114, force quantity analyzer 115, detection force quantity output unit 116, comparator 117, distal end force quantity setting circuitry 118, and flexible tube characteristic controller 119, may be included in a control device that is separate from the control device 100. For example, the shape calculator 114, force quantity analyzer 115, detection force quantity output unit 116, comparator 117, distal end force quantity setting circuitry 118, and flexible tube characteristic controller 119 may be included in a control device that is separate from an endoscope video image processor including the image processor 111 and display controller 112. Alternatively, the shape calculator 114, force quantity analyzer 115, detection force quantity output unit 116, comparator 117, distal end force quantity setting circuitry 118, and flexible tube characteristic controller 119 may be included in different control devices. Specifically, processors or hardware circuits, which function as the components of the control device 100, in particular, the shape calculator 114, force quantity analyzer 115, detection force quantity output unit 116, comparator 117, distal end force quantity setting circuitry 118, and flexible tube characteristic controller 119, may be included in a housing or housings, as far as the processors or hardware circuits can implement the functions of the components.

The image processor 111 executes a process of converting an electrical signal, to which light from a subject is converted by the imaging element 17 of the endoscope 10, to a video signal. The display controller 112 controls the operation of the display 50.

The coil controller 113 includes a coil output unit configured to output a voltage that is applied to each source coil 72 of the source coil array 73, and controls the voltage that is applied to each source coil 72 from the coil output unit.

The shape calculator 114 calculates position coordinates of each source coil 72, based on a detection signal of a magnetic field of each source coil 72 that is received by the antenna 74. Specifically, based on state information acquired from each source coil 72 and antenna (state acquisition element), the shape calculator 114 calculates bend shape information of the insertion section 11. The shape calculator 114 includes a reception unit configured to receive a detection signal from the antenna 74.

Based on the bend shape information calculated by the shape calculator 114, the force quantity analyzer 115 calculates an analysis force quantity based on a deformation force quantity (to be described later). The detection force quantity output unit 116 receives and outputs a detection signal of external force that the force quantity sensor 60 provided at the distal end of the bendable section 13 detects. The comparator 117 compares the detection force quantity from the detection force quantity output unit 116 and the analysis force quantity by the force quantity analyzer 115. The distal end force quantity setting circuitry 118 sets a distal end pushing force quantity, based on the comparison result by the comparator 117. The flexible tube characteristic controller 119 includes an output unit configured to output a control signal to a flexible tube characteristic changer 80 (to be described later), and controls the control signal that is output to the flexible tube characteristic changer 80.

In the present embodiment, each source coil 72 of the source coil array 73, the antenna 74, and the coil controller 113 and shape calculator 14 of the control device 100 constitute the bend shape detection device 71. The bend shape detection device 71, functioning as the shape acquisition device 70, detects a magnetic field generated by each source coil 72 of the source coil array 73 and calculates the bend state of the insertion section 11, thereby to support the insertion of the insertion section 11 of the endoscope 10.

The bend shape detection device 71 functioning as the shape acquisition device 70 is not limited to this. It should suffice if the bend shape detection device can detect the bend state of the insertion section 11, and may be constituted by any one of, or in combination of, for example, sensing (fiber sensor) that utilizes a variation of light quantity (light intensity) or a variation of optical characteristics of light propagating in a light guide such as an optical fiber; sensing (electromagnetic sensor) that utilizes electromagnetic waves; sensing (ultrasonic sensor) that utilizes ultrasonic; sensing (distortion sensor) that utilizes distortion; and sensing that utilizes an X-ray absorption material.

Next, the flexible tube characteristic changer 80 will be described. As shown in FIG. 3, the flexible tube section 14 is provided with the flexible tube characteristic changer 80. In the present embodiment, the flexible tube characteristic changer 80 is a stiffness variable actuator functioning as a stiffness changer that can partly change the bending stiffness of the flexible tube section 14 at a location where the flexible tube characteristic changer 80 is provided. In FIG. 3, one flexible tube characteristic changer 80 is provided, but the number of flexible tube characteristic changers 80 is not limited to one. Specifically, a plurality of flexible tube characteristic changers 80 may be provided.

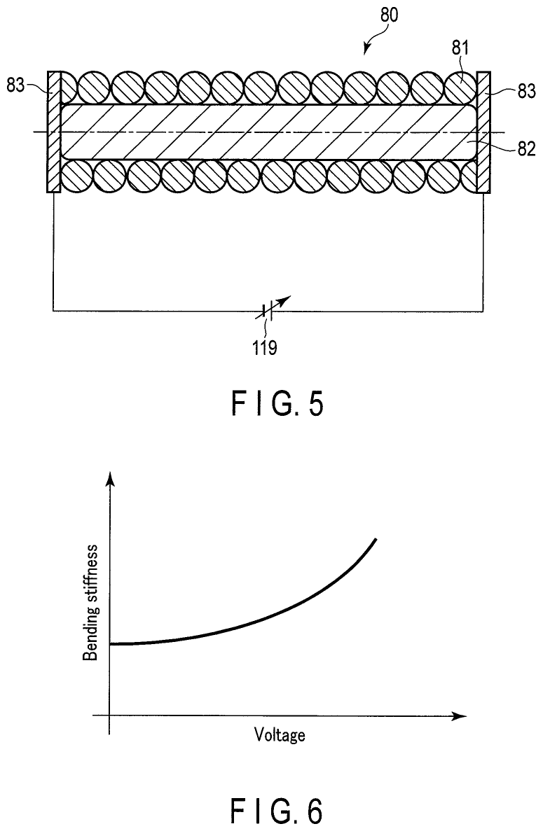

FIG. 5 is a view schematically showing an example of the flexible tube characteristic changer 80. The flexible tube characteristic changer 80 includes a coil pipe 81 composed of a metallic wire; an Electroactive Polymer Artificial Muscle (EPAM) 82 sealed in the coil pipe 81; and electrodes 83 provided at both ends of the coil pipe 81. A voltage, which is output from the flexible tube characteristic controller 119, is applied to the EPAM 82 in the coil pipe 81 through the electrodes 83. The EPAM 82 is an actuator configured to extend and contract by the application of voltage, and thereby the stiffness of the EPAM 82 changes. As shown in FIG. 6, as the applied voltage becomes higher, the bending stiffness of the flexible tube characteristic changer 80 becomes higher. Specifically, by changing the bending stiffness of the flexible tube characteristic changer 80, the bending stiffness of the flexible tube section 14 at a position where the flexible tube characteristic changer 80 is built in also changes. Accordingly, the flexible tube characteristic controller 119 applies, from an output unit thereof, a voltage to the flexible tube characteristic changer 80, and thereby the bending stiffness of the flexible tube section 14 is changed.

The input device 40 is a general input device such as a keyboard. The input device 40 is connected to the control device 100 through a cable 29. Various instructions or the like for operating the endoscope apparatus 1 are input to the input device 40. The input device 40 may be an operation panel provided on the control device 100, or a touch panel displayed on a display screen.

The display 50 is a general monitor such as a liquid crystal display. The display 50 is connected to the control device 100 through a cable 31. Based on display control by the display controller 112, the display 50 displays an endoscopic observation image that is image-processed by the image processor 111. In addition, based on the bend shape information calculated by the shape calculator 114, the display 50 may display images or character information relating to the bend shape of the insertion section 11. A display on which an endoscopic observation image is displayed, and a display on which a bend shape or the like of the insertion section 11 is displayed may be identical or may be different.

Next, a general operation of the endoscope apparatus 1 including the shape acquisition device 70 will be described.

In the endoscope apparatus 1, the insertion section 11 of the endoscope 10 is inserted into an insertion target body by a surgeon. The endoscope 10 converts light from a subject in the insertion target body to an electrical signal by the imaging element 17 of the distal end hard section 12. Then, the electrical signal is transmitted to the control device 100. The image processor 111 of the control device 100 acquires the electrical signal, and executes a process of converting the acquired electrical signal to a video signal. The display controller 112 of the control device 100 causes the display 50 to display an endoscopic observation image based on the video signal.

During the insertion, the coil controller 113 of the control device 100 applies a voltage to each source coil 72 from the coil output unit thereof. Thereby, each source coil 72 generates a weak magnetic field therearound. Specifically, information relating to the position of each source coil 72 is output from each source coil 72. The antenna 74 detects the magnetic field generated by the source coil 72, and outputs a detection signal to the shape calculator 114.

The shape calculator 114 receives the detection signal from the antenna 74 by the reception unit thereof, and calculates the bend shape of the insertion section 11 based on the detection signal. Based on the bend shape calculated by the shape calculator 114, the display controller 112 generates a three-dimensional (3D) image corresponding to the bend shape, and causes the display 50 to display the 3D image. The surgeon continues the insertion or performs a treatment, while confirming the image or character information relating to the bend shape or the endoscopic observation image, which is displayed on the display 50.

Hereinafter, a description is given of one of scope insertion methods in a colonoscopic examination, that is, a method (hereinafter referred to as “colon shortening insertion method”) in which the colon (intestinal tract) is shortened while the longitudinal axis of the insertion section 11 is being kept. Examples of the method in which the intestinal tract is shortened while the longitudinal axis of the insertion section 11 is being kept include an axis-keeping shortening method, “Hooking the fold”, and “Right turn shortening”. In the description below, it is assumed that the endoscope 10 of the endoscope apparatus 1 is a colonoscope.

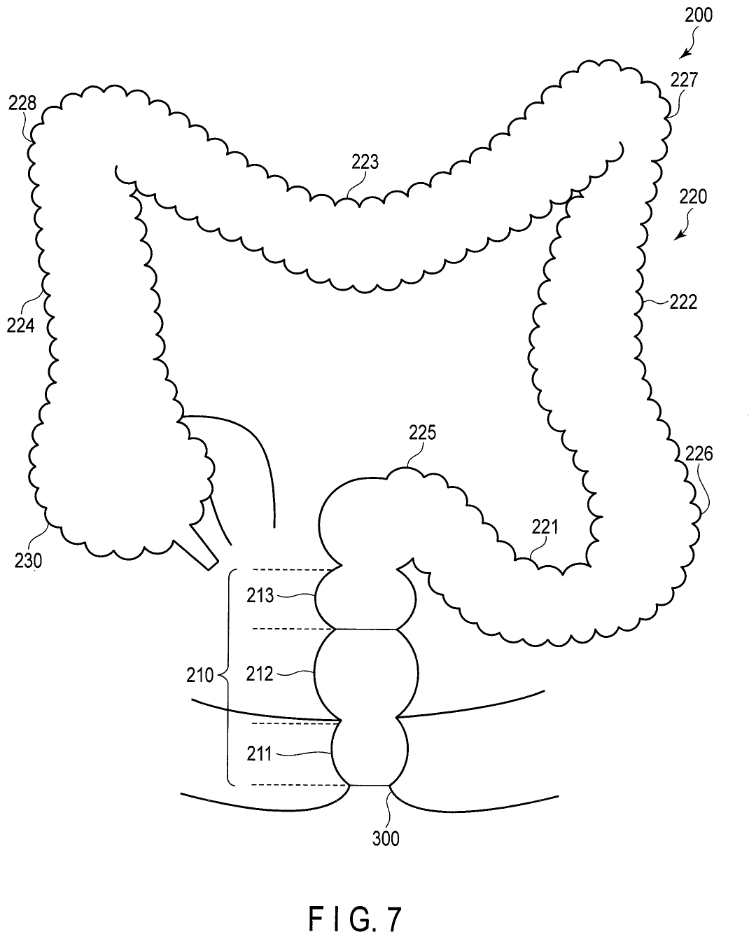

Before describing the colon shortening insertion method, the parts of the large intestine 200 will be described. FIG. 7 is an anatomical drawing schematically showing the parts of the large intestine 200. The large intestine 200 includes the rectum 210 communicating with the anus 300; the colon 220 communicating with the rectum 210; and the cecum 230 communicating with the colon 220. The rectum 210 includes the lower rectum 211, upper rectum 212, and rectal sigmoid 213 in the order from the anus side. The colon 220 includes the sigmoid colon 221, descending colon 222, transverse colon 223, and ascending colon 224 in the order from the rectum 210 side. An uppermost part of the sigmoid colon 221 is the top of sigmoid colon (so-called S-top) 225. A boundary part between the sigmoid colon 221 and descending colon 222 is the sigmoid-descending colon junction (so-called SD-Junction (SD-J)) 226. A boundary part between the descending colon 222 and transverse colon 223 is the splenic flexure (SF) 227. A boundary part between the transverse colon 223 and ascending colon 224 is the hepatic flexure (HF) 228. The S-top 225, SD-J 226, SF 227, and HF 228 are bent parts in the colon 220. The lower rectum 211 and upper rectum 212 of the rectum 210, and the descending colon 222 and ascending colon 224 of the colon 220 are fixed intestinal tracts. On the other hand, the rectal sigmoid 213 of the rectum 210, the sigmoid colon 221 and transverse colon 223 of the colon 220, and the cecum 230 are movable intestinal tracts. Specifically, the rectal sigmoid 213, sigmoid colon 221, transverse colon 223, and cecum 230 are not fixed in the abdominal part, and have movability.

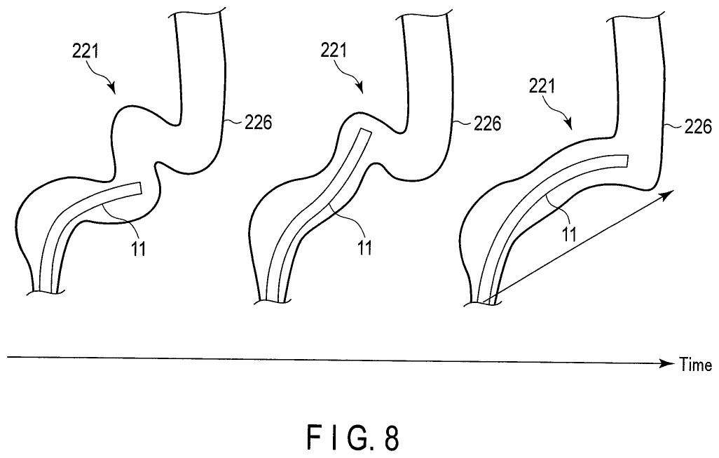

FIG. 8 is a schematic view showing an example of insertion of a colonoscope by a colon shortening insertion method. In a general scope insertion method (so-called “loop insertion method”) that is not shown, the surgeon advances the insertion section 11 by a PUSH operation that pushes the insertion section 11 from the near side along a bend shape of the intestinal tract as shown in FIG. 7. In the loop insertion method, since the insertion is advanced mainly by the PUSH operation, there is a tendency that the insertion section 11 pushes and extends the bend part (e.g. S-top 225) of the intestinal tract and pain is caused to the patient. On the other hand, in the colon shortening insertion method, the surgeon advances the insertion section 11 while telescoping the intestinal tract, without extending the intestinal tract. For example, as shown in FIG. 8, in the sigmoid colon insertion of the colonoscope, the insertion section 11 is inserted into the sigmoid colon 221 while the insertion section 11 pushes its way through the intestinal tract of the sigmoid colon 221, with the intestinal tract of the sigmoid colon 221 and the axial direction of the insertion section 11 being kept in a substantially straight shape. In the colon shortening insertion method, the surgeon does not insert the insertion section 11 by only the PUSH operation that pushes the insertion section 11 from the near side. The surgeon combines the PUSH operation and a PULL operation that pulls the insertion section 11 toward the near side, and shortens the intestinal tract by carefully pulling the intestinal tract to the near side while the distal end of the insertion section 11 is pushing its way through the intestinal tract. In particular, in the sigmoid colon insertion shown in FIG. 8, the surgeon shortens the intestinal tract along the longitudinal axis of the insertion section 11, and inserts the distal end of the insertion section 11 toward the SD-J 226 (in a direction of an arrow indicated in the right part of FIG. 8).

Thus, the colon shortening insertion method is a method in which the insertion section 11 is slowly inserted, without applying a load on the intestinal tract. Since this method can reduce the pain of the patient due to the extension of the intestinal tract, this method is known as an insertion method with a smaller load on the patient.

In the colon shortening insertion method, in order to advance the insertion section 11, it is important to slip the distal end of the insertion section 11 into a lumen beyond a bent part. For this purpose, in the colon shortening insertion method, in addition to the above-described PUSH operation and PULL operation, the surgeon performs an angle operation of the bendable section 13 by operating the angle knob 22 of the control section 21.

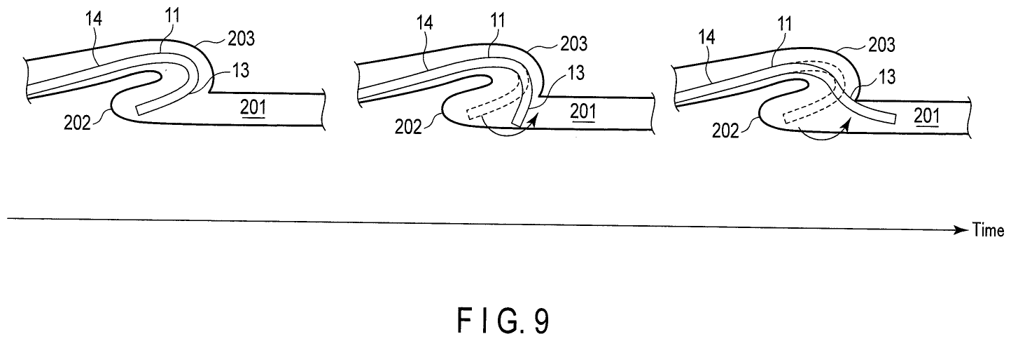

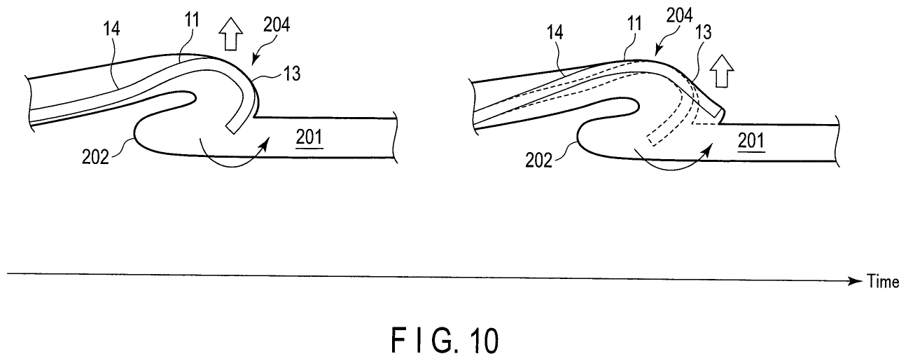

However, in the bent part of a movable intestinal tract such as the sigmoid colon 221, it is not easy to slip the distal end of the insertion section 11 into a lumen beyond the bent part. Theoretically, as shown in FIG. 9, by the surgeon performing the angle operation of the bendable section 13, it is possible to slip the distal end of the insertion section 11 into a lumen 201 beyond a bent part 202, without the insertion section 11 extending the intestinal wall 203. Specifically, by the angle operation, a bent state (a stick shape shown in a left part of FIG. 9) in which the bendable section 13 is bent along the bend shape of the bent part 202 is restored to a non-bent state (a central and right part of FIG. 9). Thereby, the distal end of the insertion section 11 can be slipped into the lumen 201. However, for example, as shown in FIG. 10, if an attempt is made to slip the distal end of the insertion section 11 into the lumen 201 by only the angle operation of the bendable section 13, a movable intestinal tract 204 may move along with the bending of the bendable section 13 by the angle operation, and the distal end of the insertion section 11 may not slip into the lumen 201 beyond the bent part 202.

Thus, in the colon shortening insertion method, the surgeon exactly slips the distal end of the insertion section 11 into the lumen 201 beyond the bent part 202, by combining the angle operation of the bendable section 13 by the angle knob 22 of the control section 21 with a twist operation that twists the insertion section 11 in a rightward direction (clockwise direction) from the near side.

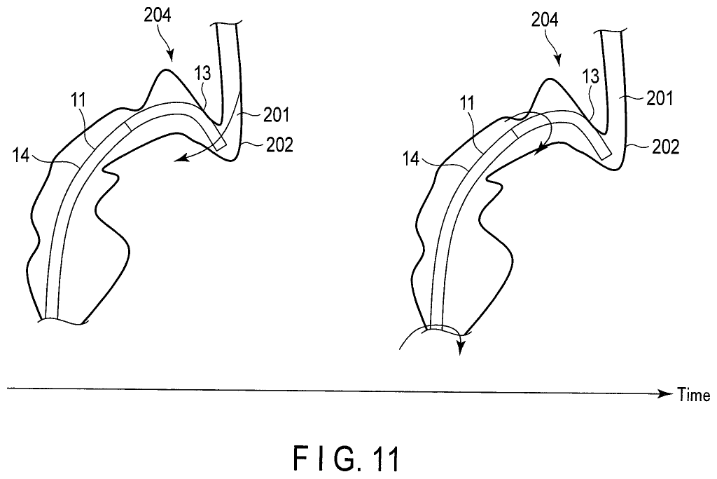

FIG. 11 is a view for explaining a concrete therapy technique in the insertion of a colonoscope by the colon shortening insertion method. As described above, with respect to the movable intestinal tract 204, it is difficult to slip the distal end of the insertion section 11 into the lumen 201 beyond the bent part 202 by only the angle operation. Thus, the surgeon performs the twist operation after the angle operation, and pushes the distal end of the insertion section 11 on the movable intestinal tract 204 in a direction in which the distal end of the insertion section 11 is to be slipped. Thereby, the movable intestinal tract 204 is kept immovable. For example, in the case of a state (straight state) in which the bendable section 13 is not angled, even if the twist operation is performed, there occurs no force that pushes the distal end of the insertion section 11 on the movable intestinal tract 204 in a direction of twisting. Thus, it is necessary to combine the angle operation and the twist operation. Specifically, in the state (angle state) in which the bendable section 13 is angled, a twist operation is performed toward the direction of the next lumen. Thereby, the distal end of the insertion section 11 is pushed on the movable intestinal tract 204. Thereafter, by restoring the bendable section 13 from the angle state to the straight state, the distal end of the insertion section 11 can exactly be slipped into the next lumen.

In this manner, in the colon shortening insertion method, for example, if the insertion section 11 catches a lumen, the surgeon slowly performs a PUSH operation in order to advance the insertion section 11 toward the direction of the next lumen. Then, with respect to the direction of the next bent lumen, the surgeon combines the angle operation of the bendable section 13 and the twist operation of the insertion section 11 as described above, and slips the distal end of the insertion section 11 toward the direction of the lumen of the intestinal tract. By repeating this, the insertion of the insertion section 11 is advanced.

In the above-described therapy technique, when the bendable section 13 restores from the angle state to the straight state, if the force that pushes the distal end of the insertion section 11 in the direction of the lumen into which the distal end of the insertion section 11 is to be slipped is weak, it is difficult to slip the distal end of the insertion section 11 into the lumen 201. Accordingly, it is necessary that a proper pushing force (distal end pushing force quantity) that is enough to slip the distal end of the insertion section 11 into the lumen 201 be generated at the distal end of the insertion section 11.

The distal end pushing force quantity of the insertion section 11 is directly proportional to the bending stiffness value of the insertion section 11 (flexible tube section 14). Thus, in the insertion section 11, for example, when the bending stiffness of the flexible tube section 14 is low, the force that pushes the intestinal tract by the distal end of the insertion section 11 becomes weak. In this case, when the bendable section 13 restores from the angle state to the straight state, the flexible tube section 14 flexes, and it becomes difficult for the distal end of the insertion section 11 to slip into the lumen 201. In addition, when the bending stiffness of the flexible tube section 14 is high, the flexible tube section 14 does not flex and the force that pushes the intestinal tract by the distal end of the insertion section 11 becomes strong. Accordingly, the distal end of the insertion section 11 easily slips into the lumen. However, if the bending stiffness value of the flexible tube section 14 is high, since the flexible tube section 14 does not easily flex, this may lead to a hindrance to the insertion in other bent parts of the intestinal tract.

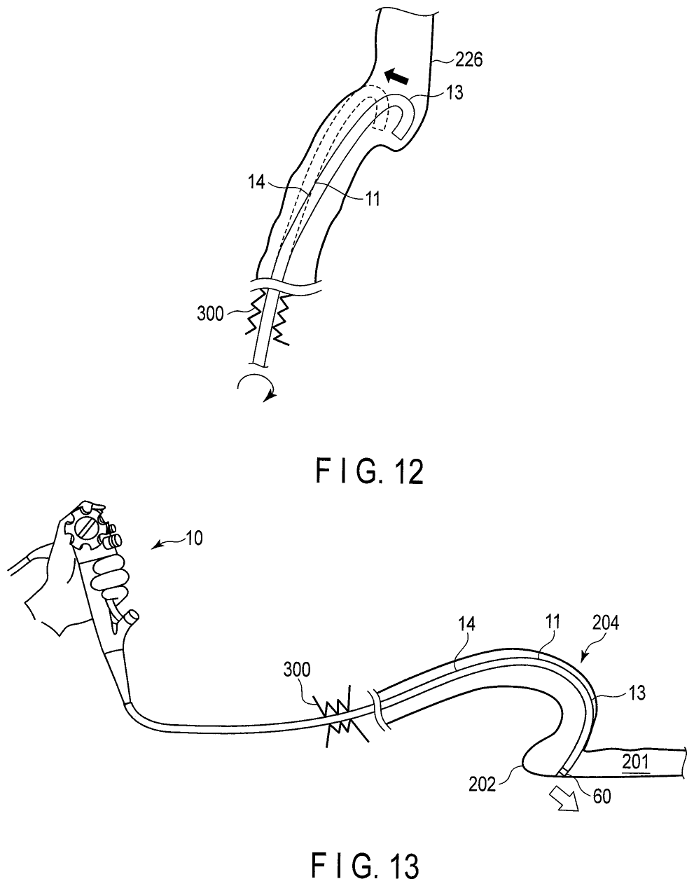

Besides, in the colon shortening insertion method, as described above, the insertion section 11 is advanced in an oblique direction toward the SD-J. At this time, as shown in FIG. 12, if the bending stiffness of the entire flexible tube section 14 is high, the distal end of the insertion section 11 shifts from the direction of the SD-J toward the center of the body cavity by a repulsive force generated by the bending stiffness (a direction of an arrow indicated near the distal end of the insertion section in the Figure). Normally, in order to slip the distal end of the insertion section 11 into the next lumen, it is preferable that the insertion section 11 is set in a state indicated by a solid line in FIG. 12. However, the insertion section 11 is set in a state indicated by a broken line. In this state, the direction of the distal end of the insertion section 11 becomes different from the direction of the SD-J.

Taking the above into account, keeping the insertion direction (advancement direction) of the insertion section 11 to be the SD-J direction and securing the distal end pushing force quantity of the insertion section 11 will lead to the colonoscope insertion support in the colon shortening insertion method. In the present embodiment, as will be described below, while the force quantity that is necessary for the distal end of the insertion section 11 when the distal end of the insertion section 11 is bent and pushed on the intestinal tract is secured, the insertion direction of the insertion section 11 is easily kept to be the direction toward the SD-J 226. Thereby, the flexible tube insertion apparatus, insertion control apparatus, and flexible tube insertion support method according to the present embodiment provide support in the colonoscope insertion by the colon shortening insertion method.

FIG. 13 is a view showing an example of colonoscope insertion in the present embodiment. FIG. 13 illustrates a state in which the distal end of the insertion section 11 of the endoscope 10 is inserted from the anus 300, and the distal end of the insertion section 11 is to be advanced to the lumen 201 beyond the bent part 202. The force with which the distal end of the insertion section 11 pushes the intestinal tract 204 is indicated by an arrow in FIG. 13. This force corresponds to the detection force quantity that is detected by the force quantity sensor 60 provided at the distal end of the insertion section 11. The bendable section 13 is bent along the bend shape of the bent part 202 by the angle operation. The flexible tube section 14 bends along the shape of the movable intestinal tract 204. In order to slip the distal end of the insertion section 11 into the lumen 201 beyond the bent part 202 from the insertion state shown in FIG. 13, the flexible tube insertion apparatus and insertion control apparatus according to the present embodiment execute bending stiffness control of the flexible tube section 14, based on a flow to be described below.

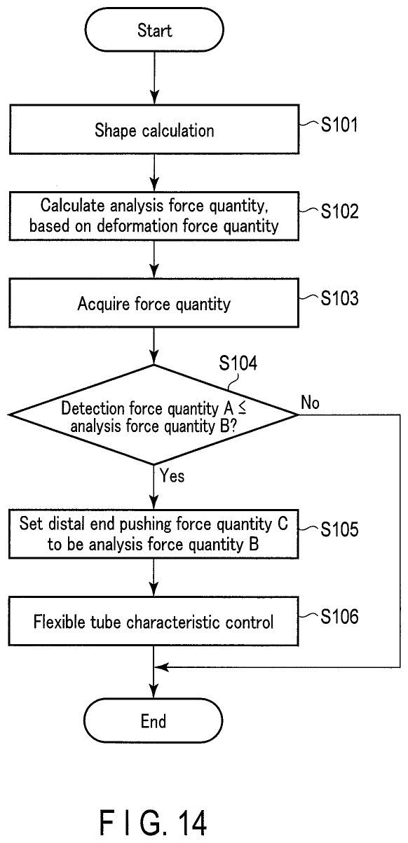

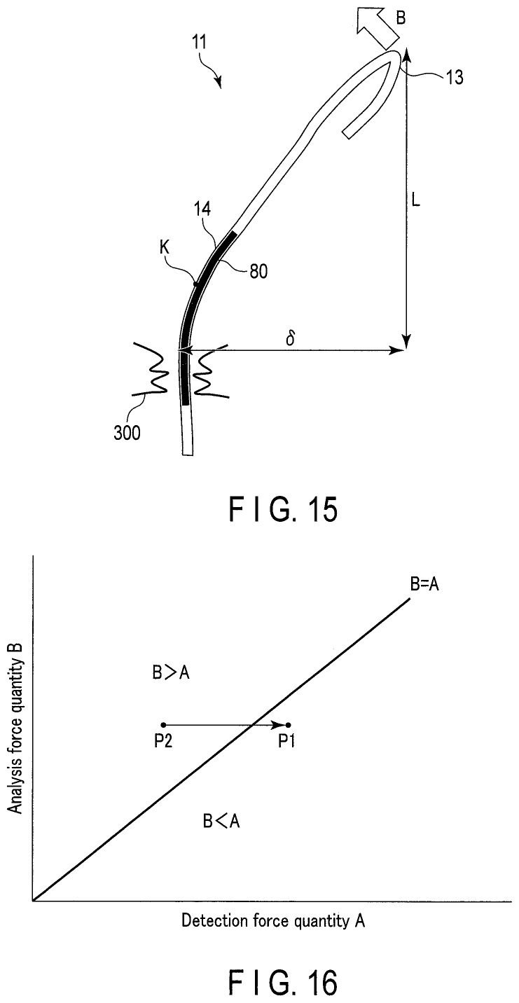

FIG. 14 is a view showing an example of an insertion support control flow in the present embodiment. In step S101, the shape calculator 114 calculates bend shape information of the insertion section 11. Specifically, the shape calculator 114 calculates the bend shape information of the insertion section 11, based on bend state information acquired from the source coils 72 and antenna 74. The bend shape information calculated here is bend shape information that is necessary for analysis of the distal end pushing force quantity, and is, for example, a length L of insertion of the insertion section 11 from the anus 300, and a bending quantity δ in an oblique direction of the insertion section 11, which are shown in FIG. 15.

In step S102, based on the bend shape information calculated in step S101, the force quantity analyzer 115 calculates an analysis force quantity based on a deformation force quantity B at a time when the insertion section 11 is deformed in a distal direction. The deformation force quantity B is a force quantity of force acting in a direction different from the axial direction of the insertion section 11, as shown in FIG. 15. For example, the deformation force quantity B is a force quantity of force acting in a direction substantially perpendicular to the distal end pushing force quantity (to be described later). The deformation force quantity B may deform the insertion section 11 near the anus 300. Specifically, according to the deformation force quantity B, as described with reference to FIG. 12, the distal end of the insertion section 11 may shift from the direction of the SD-J toward the center of the body cavity.

The deformation force quantity B is calculated by a bending stiffness value K of the insertion section 11 (flexible tube section 14), and the above-described length (insertion length) L and bending quantity δ. For example, the deformation force quantity B is calculated by

B=3·K·δ/L[N] equation (1).

The bending stiffness value K may be input to the force quantity analyzer 115 in advance.

In step S102, the force quantity analyzer 115 calculates the analysis force quantity based on the deformation force quantity B. The deformation force quantity B calculated in the above equation (1) may be set as the analysis force quantity as such. However, since there are differences in adhesion and stiffness of the intestinal tract among persons, the force quantity analyzer 115 may calculate, as the analysis force quantity, a value obtained by multiplying the deformation force quantity by a constant (0.8 to 1.2). Thereby, the analysis force quantity corresponding to the ease in movement of the intestinal tract or the difficulty in movement of the intestinal tract is calculated. Further, since friction occurs between the distal end of the insertion section 11 and the intestinal wall, a friction coefficient μ of the friction may be input to the force quantity analyzer 115 and may be used for the calculation of the analysis force quantity. Hereinafter, the description will be given on the assumption that the deformation force quantity B is set as the analysis force quantity B as such.

In step S103, the detection force quantity output unit 116 receives a detection signal from the force quantity sensor 60, and outputs the detection signal to the comparator 117. Thereby, the force quantity (contact pressure) that the insertion section 11 actually receives from the intestinal tract at the distal end of the bendable section 13 is obtained. Hereinafter, this force quantity is referred to as “detection force quantity A”.

In step S104, the comparator 117 compares magnitudes of the value of the detection force quantity A, which is output from the detection force quantity output unit 116, and the value of the analysis force quantity B by the force quantity analyzer 115. Specifically, the comparator 117 judges whether the detection force quantity A, which the distal end of the insertion section 11 actually receives, exceeds the analysis force quantity B at a time when the insertion section 11 is deformed in the distal direction.

If it is judged in step S104 that the detection force quantity A is greater than the analysis force quantity B (No), the process is finished. If the detection force quantity A, which is the force quantity that the distal end of the insertion section 11 actually receives, is greater than the analysis force quantity B, the distal end pushing force quantity that is necessary for the distal end of the insertion section 11 when the distal end of the insertion section 11 is bent and pushed on the intestinal tract is sufficiently secured. Thus, even if the surgeon continues the insertion in this state, the distal end of the insertion section 11 can be slipped into the lumen 201 beyond the bent part 202. Thus, the process is finished.

On the other hand, if it is judged in step S104 that the detection force quantity A is equal to or less than the analysis force quantity B (Yes), the process advances to step S105. If the detection force quantity A, which the distal end of the insertion section 11 actually receives, is equal to or less than the analysis force quantity B, it is considered that the distal end pushing force quantity that is necessary for the distal end of the insertion section 11 when the distal end of the insertion section 11 is bent and pushed on the intestinal tract is not sufficient. Thus, if the surgeon continues the insertion in this state, the distal end of the insertion section 11 is not easily slipped into the lumen 201 beyond the bent part 202.

Thus, in step S105, the distal end force quantity setting circuitry 118 sets a distal end pushing force quantity C that is a target value of the detection force quantity A detected by the force quantity sensor 60 to be the analysis force quantity B analyzed in step S102. Specifically, when the detection force quantity A is equal to or less than the analysis force quantity B (step S104-Yes), the distal end force quantity setting circuitry 118 sets the distal end pushing force quantity C that is the pushing force quantity by which the distal end of the insertion section 11 pushes the intestinal tract to be the analysis force quantity B by the force quantity analyzer 115.

FIG. 16 is a view showing an example of the relationship between the detection force quantity A and analysis force quantity B. In this Figure, if a point P1 that represents the case in which the detection force quantity A is greater than the analysis force quantity B (step S104-No) is plotted, the point P1 is included in a first region on or below a straight line B=A. In other words, the first region is a region corresponding to the case in which the force quantity that is necessary for the distal end of the insertion section 11 when the distal end of the insertion section 11 is bent and pushed on the intestinal tract can be obtained. In addition, if a point P2 that represents the case in which the detection force quantity A is equal to or less than the analysis force quantity B (step S104-Yes) is plotted, the point P2 is included in a second region above the straight line B=A. In other words, the second region is a region corresponding to the case in which the distal end pushing force quantity that is necessary when the distal end of the insertion section 11 is bent and pushed on the intestinal tract cannot sufficiently be obtained.

That the distal end force quantity setting circuitry 118 sets the distal end pushing force quantity C, which is the target value of the detection force quantity A, to be the analysis force quantity B in step S105 corresponds to the movement of the point P2 included in the second region to the point P1 included in the first region in FIG. 16. Specifically, by setting the distal end pushing force quantity C, the distal end pushing force quantity that is necessary when the distal end of the insertion section 11 in the endoscope 10 is bent and pushed on the intestinal tract can sufficiently be obtained.

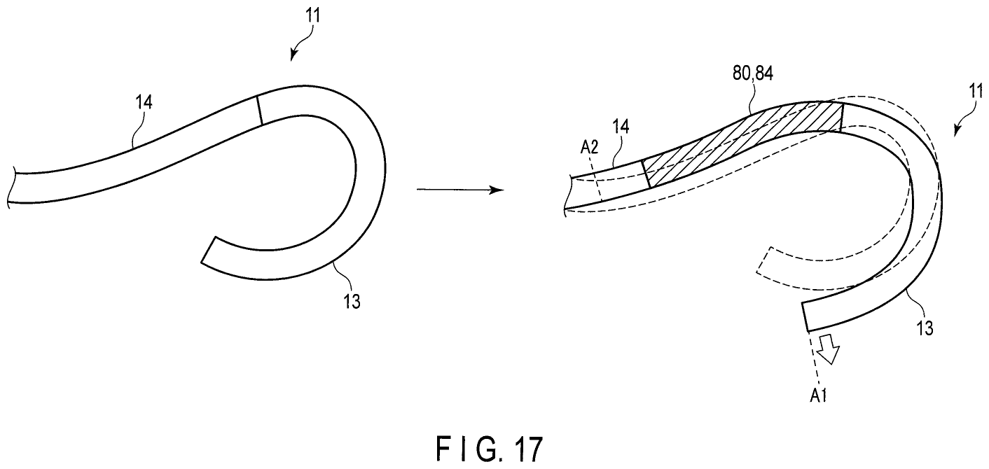

In step S106, the flexible tube characteristic controller 119 controls the bending stiffness of the flexible tube section 14 of the insertion section 11 by the flexible tube characteristic changer 80 that is a stiffness changer in the present embodiment. The flexible tube characteristic controller 119 controls the bending stiffness of the insertion section 11 by driving the flexible tube characteristic changer 80 until the value of the detection force quantity A detected by the force quantity sensor 60 becomes greater than the distal end pushing force quantity C, which is the target value of the detection force quantity A. For example, as shown in FIG. 17, the flexible tube characteristic controller 119 changes the bending stiffness such that the bending stiffness of a region 84 where the flexible tube characteristic changer 80 is provided in the flexible tube section 14 becomes high. Thereby, since the flexible tube characteristic changer 80 (flexible tube section 14) is made less easily deformable, the detection value of the force quantity sensor 60 increases. After step S106, the process is finished.

As illustrated in FIG. 17, it is preferable that the flexible tube characteristic changer 80 is included in a length of 40 cm or less from a distal end position A1 of the insertion section 11 including the distal end hard section 12, i.e. in a section from the distal end position A1 to a position A2 on the proximal end side with respect to the distal end position A1. In the colonoscope according to the present embodiment, the flexible tube characteristic changer 80 is disposed over a predetermined length in a section of 30 cm or less from the distal end of the insertion section 11 including the distal end hard section 12. For example, the flexible tube characteristic changer 80 is disposed in a section from a distal end portion of the flexible tube section 14 to a point of 30 cm from the distal end position A1 of the insertion section 11 including the distal end hard section 12.

In the case of the colonoscope, a necessary length for the insertion section 11 to pass through the sigmoid colon 221 is approximately 40 cm. If the length is greater than this, the distal end of the insertion section 11 would enter the descending colon 222 from the anus 300, and this does not suit the object of the present embodiment, i.e. to generate the distal end pushing force quantity that is necessary for the distal end of the insertion section 11 to pass through the bent part (e.g. SD-J 226). Although the passage through the sigmoid colon 221 (e.g. SD-J 226) is assumed in this embodiment as described above, the embodiment is also applicable to the SF 227 or HF 228 as a matter of course. In this case, too, if the flexible tube characteristic changer 80 is disposed at a position of greater than 40 cm from the distal end position A1, such an undesirable effect as excessive extension may be caused on other bent parts. Thus, the position of the flexible tube characteristic changer 80 is set within 40 cm from the distal end position A1.

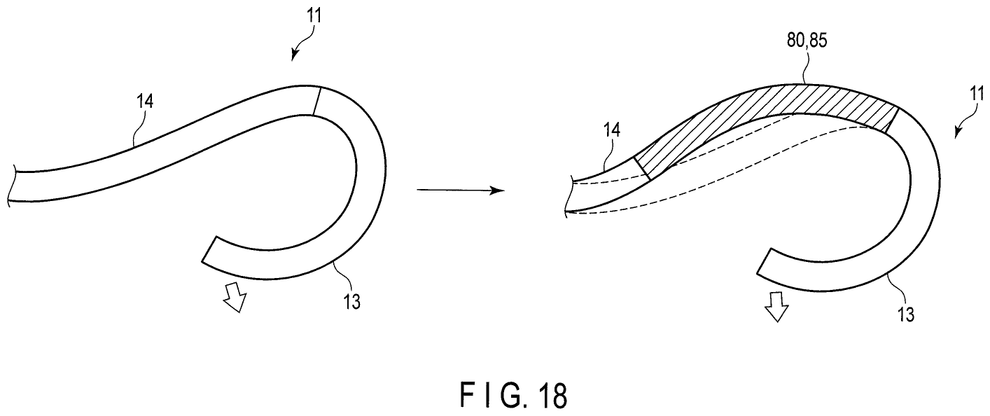

In the above description, although the flexible tube characteristic changer 80 is the stiffness variable actuator, the flexible tube characteristic changer 80 may be, for example, an actuator formed of a shape memory alloy. By using as the flexible tube characteristic changer 80 the shape memory alloy that bends in a predetermined direction upon application of heat, the flexible tube section 14 can be bent in a predetermined direction by ON/OFF control of electric power to the flexible tube characteristic changer 80 by the flexible tube characteristic controller 119. In this case, as shown in FIG. 18, in a region 85 where the flexible tube characteristic changer 80 formed of the shape memory alloy is provided, the bend shape can be moved in the angle direction of the bendable section 13. By this, too, the distal end pushing force quantity of the insertion section 11 increases.

According to the present embodiment, there can be provided a flexible tube insertion apparatus and an insertion control apparatus in which, in the colon shortening insertion method, while the force quantity that is necessary when the distal end of the insertion section 11 is bent and pushed on the intestinal tract is secured, the insertion section 11 is easily kept in an oblique direction with respect to the SD-J. For example, in a colonoscopic examination, it is difficult to master sigmoid colon insertion. In particular, it is difficult for an inexperienced surgeon to slip a distal end of an insertion section into the next lumen existing beyond a bent part of a movable intestinal tract. However, according to the present embodiment, since the flexible tube insertion apparatus or insertion control apparatus provides proper insertion support, the surgeon's operation can be made easier. Moreover, since the flexible tube insertion support method such as an insertion support control flow according to the embodiment is executed by a computer such as a CPU, proper insertion support can be provided.

After the flexible tube characteristic controller 119 turns on the bending stiffness control of the flexible tube characteristic changer 80 in step S106, the control device 100 detects that the distal end of the insertion section 11 has slipped into the lumen 201 beyond the bent part 202, and the flexible tube characteristic controller 119 may turn off the bending stiffness control of the flexible tube characteristic changer 80.

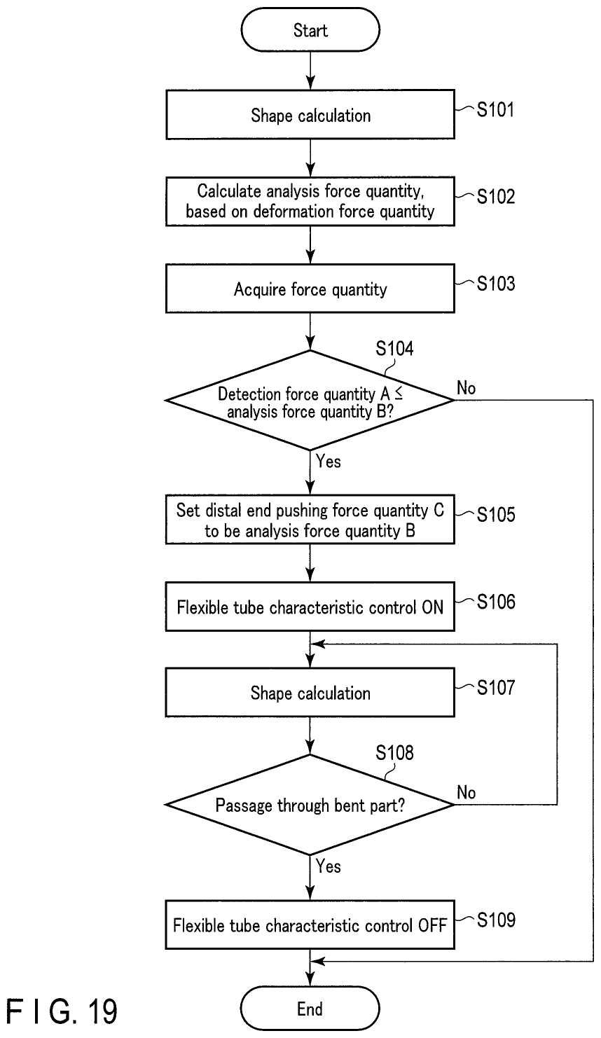

FIG. 19 is a view showing another example of the insertion support control flow according to the present embodiment. Steps S101 to S106 are the same as described above. In step S106, the flexible tube characteristic controller 119 controls the bending stiffness of the flexible tube section 14 of the insertion section 11 by the flexible tube characteristic changer 80 (flexible tube characteristic control ON). The flexible tube characteristic controller 119 increases the bending stiffness of the insertion section 11 by driving the flexible tube characteristic changer 80 until the value of the detection force quantity A detected by the force quantity sensor 60 becomes greater than the distal end pushing force quantity C that is the target value of the detection force quantity A.

In step S107, the shape calculator 114 calculates the bend shape of the insertion section 11. In step S108, based on the bend shape calculated in step S107, the control device 100 judges whether the insertion section 11 has passed through the bent part 202. If the insertion section 11 has passed through the bent part 202, for example, the bendable section 13 is in a straight state.

If it is judged in step S108 that the insertion section 11 has not passed through the bent part 202 (No), the process returns to step S107. On the other hand, if it is judged that the insertion section 11 has passed through the bent part 202 (Yes), the process advances to step S109. In step S109, the flexible tube characteristic controller 119 turns off the bending stiffness control of the flexible tube characteristic changer 80 (flexible tube characteristic control OFF), and the bending stiffness value of the insertion section 11 is restored to the original value. Then, the process is finished.

For example, if the angle knob 22 of the control section 21 is provided with an encoder configured to detect an operation quantity of the angle knob 22, it is possible to detect that the bendable section 13 is in the angle state or in the straight state, based on the output from the encoder. Accordingly, instead of steps S107 and S108, it may be judged, based on the angle operation quantity, whether the insertion section 11 has passed through the bent part 202.

By this insertion support control flow, too, the operation of the surgeon can be made easier. In particular, by the flexible tube characteristic control ON/OFF, proper insertion support can be provided at a time of the passage through a plurality of bent parts.

### Second Embodiment

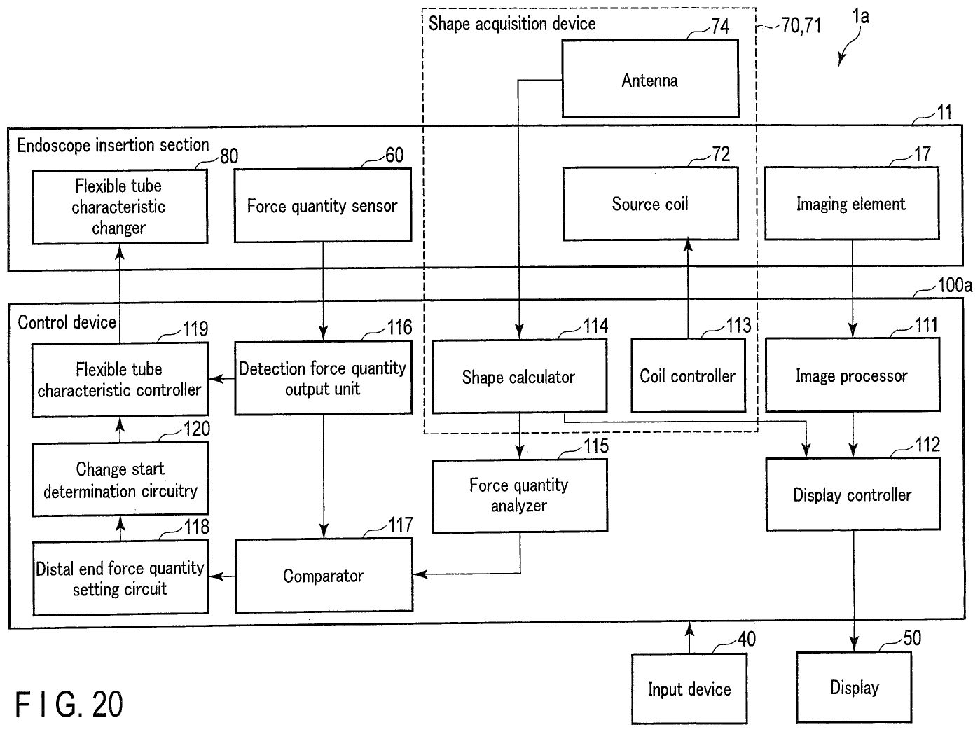

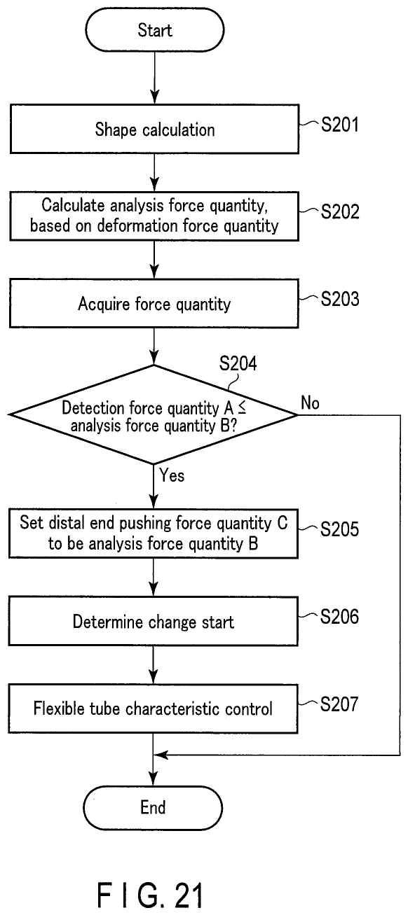

A second embodiment of the present invention will be described with reference to FIG. 20 and FIG. 21. In the description below, different parts from the first embodiment will be mainly described. Similar structures or the like to the first embodiment are denoted by the same reference signs as in the first embodiment, and a description thereof is omitted.

FIG. 20 is a block diagram showing an example of an endoscope apparatus 1a according to a second embodiment. The endoscope apparatus 1a includes an endoscope 10 (FIG. 20 shows only an endoscope insertion section 11), a light source device 30, an input device 40, a display 50, and a control device 100a. In FIG. 20, a light source device 30 is omitted. In the present embodiment, the control device 100a includes a change start determination circuitry 120, in addition to the respective components of the control device 100 in the first embodiment. Like each component of the control device 100a, the change start determination circuitry 120 may be composed of a processor such as a CPU, or an ASIC, FPGA or the like. The change start determination circuitry 120 determines a control timing of the flexible tube characteristic changer 80 by the flexible tube characteristic controller 119. For example, a function of control ON/OFF of the flexible tube characteristic changer 80 by the flexible tube characteristic controller 119 is assigned to the switch 24 (see FIG. 1) of the control section 21. Specifically, by pressing the switch 24 to which this function is assigned, the surgeon can execute control ON/OFF of the flexible tube characteristic change. The change start determination circuitry 120 may be included in a control device separate from the control device 100, as far as the function of the change start determination circuitry 120 can be implemented.

FIG. 21 is a view showing an example of an insertion support control flow in the present embodiment. Steps S201 to S205 are the same as steps S101 to S105 in the first embodiment. Specifically, in step S201, the shape calculator 114 calculates bend shape information of the insertion section 11. In step S202, the force quantity analyzer 115 calculates the analysis force quantity B, based on the deformation force quantity at a time when the insertion section 11 is deformed in the distal direction. In step S203, the detection force quantity output unit 116 acquires the detection force quantity A from the force quantity sensor 60. In step S204, the comparator 117 compares the magnitudes of the value of the detection force quantity A and the value of the analysis force quantity B. If it is judged in step S204 that the detection force quantity A is greater than the analysis force quantity B (No), the process is finished. If it is judged in step S204 that the detection force quantity A is equal to or less than the analysis force quantity B (Yes), the process advances to step S205. In step S205, the distal end force quantity setting circuitry 118 sets the distal end pushing force quantity C to be the analysis force quantity B.

In step S206, the change start determination circuitry 120 determines control ON/OFF of the flexible tube characteristic changer 80, based on the presence/absence of a control signal by the doctor's pressing of the switch 24. In step S206, the change start determination circuitry 120 stands by for the start of bending stiffness control of the flexible tube characteristic changer 80 until receiving the control signal. Upon receiving the control signal, the process advances to step S207.

In step S207, the flexible tube characteristic controller 119 controls the bending stiffness of the flexible tube section 14 of the insertion section 11 by the flexible tube characteristic changer 80. The flexible tube characteristic controller 119 controls the bending stiffness of the insertion section 11 by driving the flexible tube characteristic changer 80 until the value of the detection force quantity A detected by the force quantity sensor 60 becomes greater than the distal end pushing force quantity C that is the target value of the detection force quantity A. Thereby, since the flexible tube characteristic changer 80 (flexible tube section 14) is made less easily deformable, the detection value of the force quantity sensor 60 increases. After step S207, the process is finished.

In step S206, a standby time may be preset in the change start determination circuitry 120. Thereby, when the change start determination circuitry 120 does not receive a control start signal even after the passage of a predetermined time, the process may advance to step S207 and then the process may be finished.

According to the present embodiment, since the change start determination circuitry 120 is provided, the surgeon can freely determine the change timing. Therefore, proper control based on the feeling of the surgeon's hand, experience, etc. can be executed.

### Third Embodiment

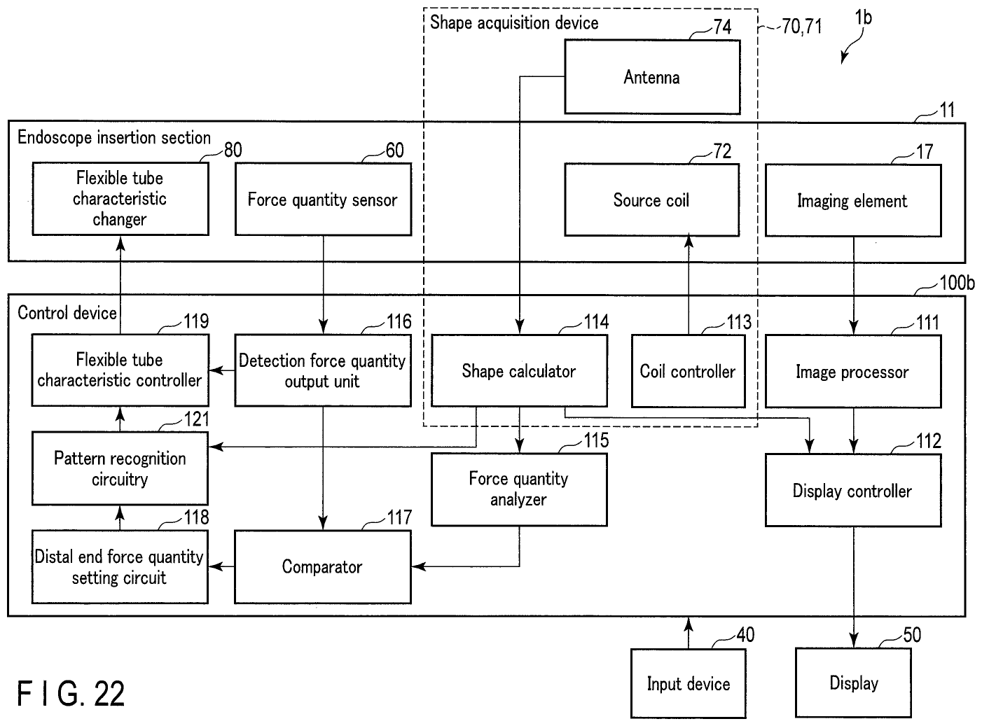

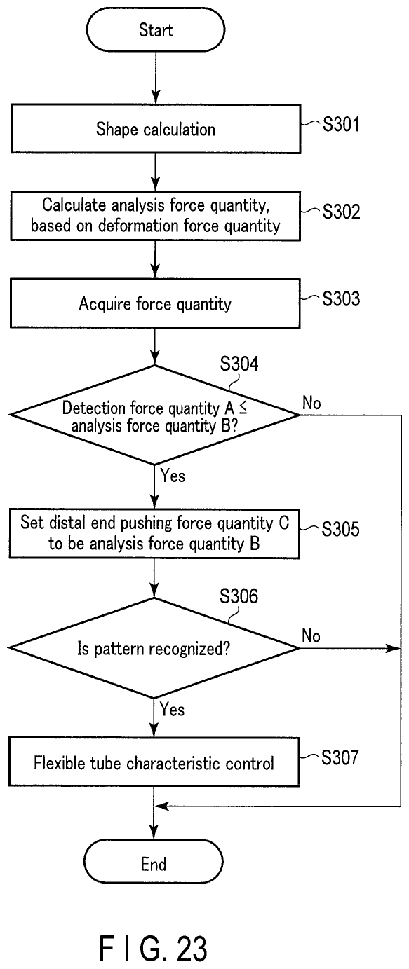

A third embodiment of the present invention will be described with reference to FIG. 22 and FIG. 23. In the description below, different parts from the first embodiment will be mainly described. Similar structures or the like to the first embodiment are denoted by the same reference signs as in the first embodiment, and a description thereof is omitted.

FIG. 22 is a block diagram showing an example of an endoscope apparatus 1b in the third embodiment. The endoscope apparatus 1b includes an endoscope 10 (FIG. 22 shows only an endoscope insertion section 11), a light source device 30, an input device 40, a display 50, and a control device 100b. In FIG. 22, a light source device 30 is omitted. In the present embodiment, the control device 100b includes a pattern recognition circuitry 121, in addition to the respective components of the control device 100 in the first embodiment. Like each component of the control device 100b, the pattern recognition circuitry 121 may be composed of a processor such as a CPU, or an ASIC, FPGA or the like. The pattern recognition circuitry 121 performs pattern recognition of the bend shape of the insertion section 11, based on the bend shape of the insertion section 11, which is calculated by the shape calculator 114, and a pattern shape of the insertion section 11, which the pattern recognition circuitry 121 prestores or which the pattern recognition circuitry 121 acquires from a storage unit (not shown) or the like. The pattern shape may be, for example, a pattern shape of the insertion section 11 in the sigmoid colon insertion by the colon shortening insertion method, a pattern shape in the transverse colon insertion, or a pattern shape in the ascending colon insertion. The pattern recognition circuitry 121 may be included in a control device separate from the control device 100, as far as the function of the pattern recognition circuitry 121 can be implemented.

FIG. 23 is a view showing an example of an insertion support control flow in the present embodiment. Steps S301 to S305 are the same as steps S101 to S105 in the first embodiment. Specifically, in step S301, the shape calculator 114 calculates bend shape information of the insertion section 11. In step S302, the force quantity analyzer 115 calculates the analysis force quantity B, based on the deformation force quantity. In step S303, the detection force quantity output unit 116 acquires the detection force quantity A from the force quantity sensor 60. In step S304, the comparator 117 compares the magnitudes of the value of the detection force quantity A and the value of the analysis force quantity B. If it is judged in step S304 that the detection force quantity A is greater than the analysis force quantity B (No), the process is finished. If it is judged in step S304 that the detection force quantity A is equal to or less than the analysis force quantity B (Yes), the process advances to step S305. In step S305, the distal end force quantity setting circuitry 118 sets the distal end pushing force quantity C to be the analysis force quantity B.

In step S306, the pattern recognition circuitry 121 performs pattern recognition by comparing the bend shape of the insertion section 11 calculated by the shape calculator 114 and a pattern shape to be referred to, which the pattern recognition circuitry 121 has. Specifically, the pattern recognition circuitry 121 judges whether or not the insertion section 11 has a pattern shape that requires bending stiffness changing control by the flexible tube characteristic controller 119 in order to apply a proper distal end pushing force. If the pattern is not recognized (No), the process is finished. If the pattern is recognized (Yes), the process advances to step S307.

In step S307, the flexible tube characteristic controller 119 controls the bending stiffness of the flexible tube section 14 of the insertion section 11 by the flexible tube characteristic changer 80. The flexible tube characteristic controller 119 controls the bending stiffness of the insertion section 11 by driving the flexible tube characteristic changer 80 until the value of the detection force quantity A detected by the force quantity sensor 60 becomes greater than the distal end pushing force quantity C that is the target value of the detection force quantity A. Thereby, since the flexible tube characteristic changer 80 (flexible tube section 14) is made less easily deformable, the detection value of the force quantity sensor 60 increases. After step S307, the process is finished.

According to the present embodiment, since the pattern recognition circuitry 121 is provided, a proper flexible tube characteristic control based on the bend shape pattern that is set can be executed. Specifically, by the flexible tube characteristic control based on the recognition of the preset bend shape pattern, proper insertion support, which does not depend on the degree of skill of the surgeon, can be provided.

### Fourth Embodiment

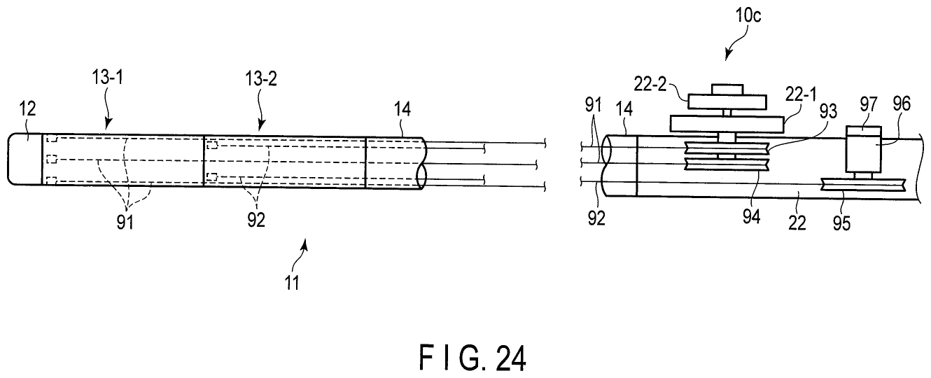

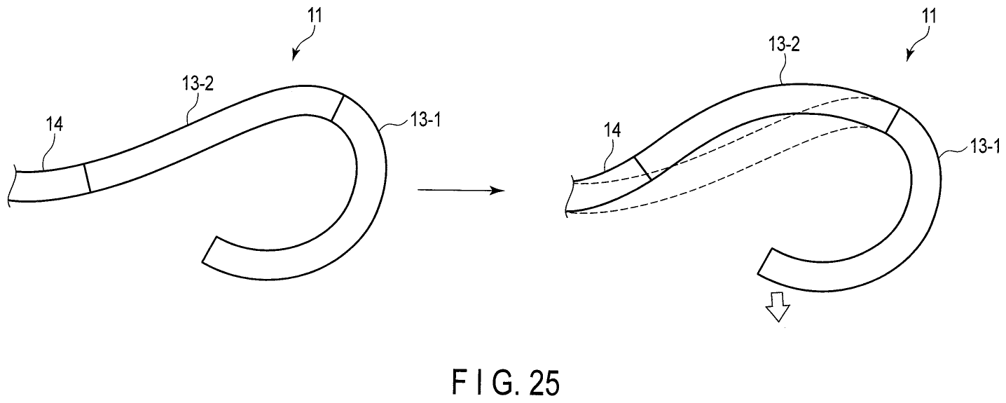

A fourth embodiment of the present invention will be described with reference to FIG. 24 and FIG. 25. In the description below, different parts from the first embodiment will be mainly described. Similar structures or the like to the first embodiment are denoted by the same reference signs as in the first embodiment, and a description thereof is omitted.

FIG. 24 is a view showing an example of an endoscope 10c in the fourth embodiment. In the fourth embodiment, an endoscope 10c, which is substituted for the endoscope 10 of the first embodiment, includes a second bendable section 13-2 configured to change mechanical characteristics of the insertion section 11 as a flexible tube characteristic changer, in addition to a first bendable section 13-1 that corresponds to the bendable section 13 of the first embodiment. Specifically, in the present embodiment, the insertion section 11 includes a distal end hard section 12, the first bendable section 13-1, the second bendable section 13-2, and a flexible tube section 14 in the order from the distal end side to the proximal end side. The second bendable section 13-2 may be regarded as a flexible tube characteristic changer, which is provided in a part of the flexible tube section 14 and changes mechanical characteristics of the flexible tube section 14.

Four angle wires 91 are fixed to a distal end of the first bendable section 13-1. Of the four angle wires 91, two angle wires 91 for bending the first bendable section 13-1 in an up-and-down direction are wound around a first drum 93 in the control section 21 and are fixed. The first drum 93 is rotated by a first angle knob 22-1 of the control section 21. By the rotation of the first angle knob 22-1, the first bendable section 13-1 bends in an upward direction or a downward direction. In addition, two angle wires 91 for bending the first bendable section 13-1 in a right-and-left direction are wound around a second drum 94 in the control section 21 and are fixed. The second drum 94 is rotated by a second angle knob 22-2 of the control section 21. By the rotation of the second angle knob 22-2, the first bendable section 13-1 bends in a left direction or a right direction.

Two angle wires 92 for bending the second bendable section 13-2 in an up-and-down direction are fixed to a distal end of the second bendable section 13-2. The angle wires 92 are connected to a motor 96 through a pulley 95 in the control section 21. By the driving force from the motor 96, the second bendable section 13-2 bends in an upward direction or a downward direction. An encoder 97 is attached to the motor 96.

In the present embodiment, the second bendable section 13-2 receives the driving force from the motor 96, and can be bent by the angle wires 92 in the up-and-down direction. Accordingly, as shown in FIG. 25, as the flexible tube characteristic control, the first bendable section 13-1 and second bendable section 13-2 can be moved in the same direction. Thereby, like the first to third embodiments, the distal end pushing force quantity of the insertion section 11 increases.

Note that, aside from the driving control by the motor 96, a manual angle operation may be performed for the second bendable section 13-2.

According to the present embodiment, like the first to third embodiments, there can be provided a flexible tube insertion apparatus, an insertion control apparatus, or a flexible tube insertion support method in which, while the force quantity that is necessary when the distal end of the insertion section 11 is bent and pushed is secured, the insertion section 11 is easily kept in an oblique direction with respect to the SD-J.

In the above description, each embodiment of the present invention has been described by being exemplified by the endoscope apparatus 1 including the colonoscope. However, the present invention is not limited to the endoscope apparatus, and includes a flexible tube insertion apparatus including a flexible insertion section.

The present invention is not limited to the above-described embodiments. In practice, various modifications can be made without departing from the spirit of the invention. The embodiments can be properly combined and implemented as much as possible and, in this case, combined advantageous effects can be obtained. Further, the embodiments include inventions of various stages, and various inventions can be extracted by proper combinations of disclosed constituent elements.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube insertion apparatus comprising:
an insertion section having a distal end and a proximal end, and including a flexible tube section configured to passively bend, and a bendable section that is connected to the flexible tube section on a distal end side of the flexible tube section and configured to actively change its bend shape, the insertion section being to be inserted into an insertion target body from the distal end side;
a flexible tube characteristic changer that is provided in the flexible tube section and configured to change a force quantity acting on the distal end of the insertion section from the insertion target body, by changing a characteristic of the flexible tube section;
a force quantity analyzer configured to calculate information relating to a deformation force quantity that is force that restores bending of the insertion section on the proximal end side with respect to the flexible tube characteristic changer; and
a flexible tube characteristic controller configured to control the flexible tube characteristic changer, based on the information relating to the deformation force quantity.

2. The flexible tube insertion apparatus according to claim 1, wherein
the force quantity analyzer is configured to calculate an analysis force quantity, based on the deformation force quantity,
the flexible tube insertion apparatus further comprises:
an external force detector that is disposed at the distal end of the insertion section and configured to detect a force quantity acting on the distal end of the insertion section;
a comparator configured to compare magnitudes of a detection force quantity by the external force detector and the analysis force quantity by the force quantity analyzer; and
a distal end force quantity setting circuitry configured to set the analysis force quantity to be a distal end pushing force quantity when the comparator judges that the analysis force quantity is greater than the detection force quantity, and
the flexible tube characteristic controller is configured to change, when the comparator judges that the analysis force quantity is greater than the detection force quantity, a characteristic of the flexible tube characteristic changer until the detection force quantity by the external detector exceeds the distal end pushing force quantity.

3. The flexible tube insertion apparatus according to claim 2, wherein
the force quantity analyzer is configured to analyze the distal end pushing force quantity, based on bend shape information of the insertion section calculated by a shape calculator based on a bend state acquired by a state acquisition element configured to acquire a bend state of the insertion section.

4. The flexible tube insertion apparatus according to claim 3, further comprising
the state acquisition element and the shape calculator.

5. The flexible tube insertion apparatus according to claim 1, further comprising
a change start determination circuitry configured to determine a start of a characteristic change of the flexible tube characteristic changer by the flexible tube characteristic controller.

6. The flexible tube insertion apparatus according to claim 3, further comprising
a pattern recognition circuitry configured to recognize a bend shape pattern of the insertion section, the flexible tube characteristic controller being configured to change the characteristic of the flexible tube characteristic changer, based on a bend pattern shape of the insertion section that is preset in the pattern recognition circuitry, and the bend shape information of the insertion section calculated by the shape calculator.

7. The flexible tube insertion apparatus according to claim 1, wherein
the flexible tube characteristic changer is a bending stiffness changer configured to change bending stiffness of the insertion section.

8. The flexible tube insertion apparatus according to claim 2, wherein
the distal end pushing force quantity is calculated by a bending stiffness value of the insertion section, an insertion length of the insertion section into the insertion target body, and a bending quantity of the insertion section in an oblique direction.

9. The flexible tube insertion apparatus according to claim 8, wherein
the distal end pushing force quantity is calculated by using a friction coefficient of friction occurring between the distal end of the insertion section and the insertion target body.

10. The flexible tube insertion apparatus according to claim 2, wherein
the force quantity analyzer is configured to calculate, as the analysis force quantity, a force quantity obtained by multiplying the deformation force quantity by a constant of 0.8 to 1.2.

11. The flexible tube insertion apparatus according to claim 1, wherein
the flexible tube characteristic changer is formed of a shape memory alloy, the shape memory alloy being capable of moving a bend shape of the flexible tube section in the same direction as a direction of bending of the bendable section.

12. The flexible tube insertion apparatus according to claim 1, wherein
the bendable section includes a first bendable section, and a second bendable section that is provided on a proximal end side of the first bendable section and constitutes the flexible tube characteristic changer, and
the second bendable section is bendable in the same direction as the first bendable section.

13. The flexible tube insertion apparatus according to claim 1, wherein
the insertion section is a part of a colonoscope, and
the flexible tube characteristic changer is disposed over a predetermined length within 40 cm or less from the distal end of the insertion section.

14. The flexible tube insertion apparatus according to claim 7, wherein
the flexible tube characteristic controller is configured to control, when the deformation force quantity is a first magnitude, the bending stiffness changer so that bending stiffness of the bending stiffness changer becomes a first bending stiffness, and is configured to control, when the deformation force quantity is a second magnitude that is greater than the first magnitude, the bending stiffness changer so that the bending stiffness of the bending stiffness changer becomes a second bending stiffness that is higher than the first bending stiffness.

15. An insertion control apparatus comprising:
a force quantity analyzer configured to calculate information relating to a deformation force quantity that is force that restores bending of an insertion section on a proximal end side with respect to a flexible tube characteristic changer, the insertion section having distal end and a proximal end, including a flexible tube section configured to passively bend, and a bendable section that is connected to the flexible tube section on a distal end side of the flexible tube section and configured to actively change its bend shape, and being to be inserted into an insertion target body from the distal end side, the flexible tube characteristic changer being provided in the flexible tube section and configured to change a force quantity acting on the distal end of the insertion section from the insertion target body, by changing a characteristic of the flexible tube section; and
a flexible tube characteristic controller configured to control the flexible tube characteristic changer, based on the information relating to the deformation force quantity.

16. A flexible tube insertion support method comprising:
inserting an insertion section into an insertion target body, the insertion section having a distal end and a proximal end, and including a flexible tube section configured to passively bend, and a bendable section that is connected to the flexible tube section on a distal end side of the flexible tube section and configured to actively change its bend shape;
changing a force quantity acting on the distal end of the insertion section from the insertion target body, by changing a characteristic of the flexible tube section; and
calculating information relating to a deformation force quantity that is force that restores bending of the insertion section on the proximal end side with respect to a location where the characteristic of the flexible tube section is changed,
the changing the force quantity acting on the distal end of the insertion section being executed based on the information relating to the deformation force quantity.
